(19) United States
(12) Patent Application Publication
(10) Pub. No.: US 2010/0228034 A1
(43) Pub. Date: Sep. 9, 2010

(54) PROCESS FOR THE PREPARATION OF KETO INTERMEDIATES

(75) Inventors: Emanuele ATTOLINO, Palagiano (TA) (IT); Lino Colombo, Pavia (IT); Ada Maria Zurlo, Mesagne (BR) (IT); Marco Artico, Parabiago (MI) (IT); Pietro Allegrini, San Donato Milanese (MI) (IT)

Correspondence Address: ROTHWELL, FIGG, ERNST & MANBECK, P.C., 1425 K STREET, N.W., SUITE 800, WASHINGTON, DC, 20005, US

(73) Assignee: DIPHARMA FRANCIS S.r.I., Baranzate (MI) (IT)

(21) Appl. No.: 12/692,719

(22) Filed: Jan. 25, 2010

(30) Foreign Application Priority Data
Mar. 4, 2009 (IT) MI2009A000311

(51) Int. Cl. C07D 211/34

(52) U.S. Cl. 546/239

(57) ABSTRACT

Process for the preparation of 4-[1-oxo-4-[4-(hydroxyphenylmethyl)-1-piperidinyl]butyl]-α,α-dimethylbenzenacetic acid, which is an intermediate useful in the preparation of fexofenadine, by hydrating asymmetric alkynes.

### TECHNICAL FIELD

The present invention relates to a novel process for the preparation of synthetic intermediates such as 4-[1-oxo-4-[4-(hydroxyphenylmethyl)-1-piperidinyl]butyl]-α,α-dimethylbenzenacetic acid 4, useful in the preparation of fexofenadine.

### BACKGROUND ART

Several processes are known for the preparation of fexofenadine, such as the ones disclosed in WO 93/21156, WO 97/22344 and WO 97/23213, characterized by many steps. None of these processes has a converging approach, but rather the building of the molecule through the consecutive introduction of the different moieties starting from α,α-dimethylbenzenacetic acid.

An alternative route of synthesis is disclosed by Kawai S. et al. in J. Org. Chem. 1994, 59, 2620-2622, but this route has several drawbacks that prevent its industrial application. A key passage of such synthetic route is the hydration of the triple bond of the methylester of formula (A) to obtain the corresponding keto derivative of formula (B),

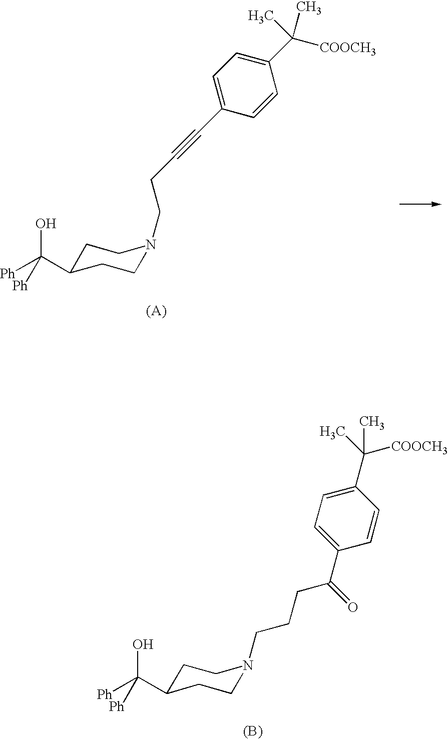

wherein Ph is a phenyl ring.

The hydration of the asymmetric alkynes catalyzed by protic acids generally results troublesome due to the high formation of regioisomers or undesired by-products.

The problem of the formation of by-products resulting from the hydration reaction of a compound of formula (A) has been partially solved by EP 1260505, where such hydration is performed by making use of a Pt, Pd or Ru metal based catalyst, optionally in presence of a ligand. A more efficient method for hydrating the triple bond to obtain the corresponding keto intermediate has been disclosed in EP 1616861 and in EP1992615 by using an Hg(II) based catalyst in methanol or in tetrahydrofuran, respectively. In particular, the catalytic system disclosed in EP 1992615 makes the process particularly regioselective and allows the isolation of the desired keto compound with high yields thanks to the reduced formation of by-products which are difficult to be removed.

J. Am. Chem. Soc., 2009, 131 (2), 448-449 discloses an efficient method for hydrating symmetric or asymmetric terminal alkynes catalyzed by Au(I) based catalysts which works in neutral conditions and with high TONs (turnover numbers). Anyway, such method is scarcely regioselective and on asymmetric inner alkynes leads to the formation of regioisomers hardly separable on industrial scale. Therefore, there is the need of an alternative process which on industrial scale affords a keto intermediate, in particular useful in the synthesis of fexofenadine, with high yields. In particular such process should allow an efficient and regioselective hydration of intermediates containing an inner asymmetric triple bond, by making use of the smallest amount of metal catalyst.

### SUMMARY OF THE INVENTION

It has now been found a novel process for the preparation of a keto compound having formula (I), as herein defined, comprising the reaction of an asymmetric alkyne of formula (II), as herein defined, with a strong protic acid, in presence of water and of an Au(I) based catalyst. The yields obtained are surprisingly very high, with a very small formation of by-products, and the catalyst can be used in amounts also lower than 1% molar. This makes the process of the invention extremely advantageous on industrial scale.

### DETAILED DESCRIPTION OF THE INVENTION

Object of the invention is a process for the preparation of a compound of formula (I)

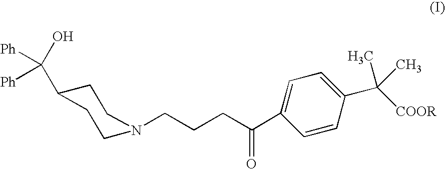

wherein Ph is a phenyl ring and R is hydrogen or a C₁-C₆ alkyl group;

comprising reacting an alkyne of formula (II)

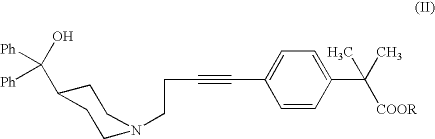

wherein Ph and R are as defined above, with a strong protic acid in presence of water and of an Au(I) based catalyst and, if desired, the conversion of a compound of formula (I) into another compound of formula (I).

A C₁-C₆ alkyl group is typically a straight or branched C₁-C₄ alkyl group, for example methyl, ethyl, propyl, isobutyl, or tert-butyl, preferably methyl.

An Au(I) based catalyst is typically an Au(I) compound having the following formula (III)

P(Ra)₃AuX (III)

wherein in the ligand P(Ra)₃ each of Ra, being the same or different, is a straight or branched C₁-C₃₀ alkyl group; a C₃-C₁₀ cycloalkyl ring; or an aryl or heteroaryl ring; and wherein X is a coordinating or non-coordinating, organic or inorganic anion.

A C₁-C₃₀ alkyl group is for example a straight or branched C₁-C₆ alkyl group, in particular methyl, ethyl, propyl, isopropyl, butyl or tert-butyl; preferably tert-butyl.

A C₃-C₁₀ cycloalkyl ring is typically a cyclopentyl, cyclohexyl or cycloheptyl ring, in particular a cyclohexyl ring.

An aryl ring is preferably a phenyl ring optionally substituted by a C₁-C₄ alkyl group, in particular methyl.

A heteroaryl ring is for example a heteromonocyclic or heterobicyclic ring, containing from 1 to 3 heteroatoms independently chosen from nitrogen, oxygen and sulphur, for example a piridyl or pirazinyl ring, preferably a piridyl ring.

Preferably the ligand P(Ra)₃ is tritert-butylphosphine, tricyclohexylphosphine, triphenylphosphine or tritolylphosphine.

A coordinating anion X can be a halide, preferably chloride or bromide. The term non-coordinating anion is meant herein to comprise also the slightly coordinating anions known in the art. Said anion can for example be an azide; sulphate; nitrate; sulfonate for example mesylate, tosylate or trifluoromethanesulfonate, preferably trifluoromethanesulfonate; sulphinate; C₁-C₆ alcoholate; phenate; carboxylate; perchlorate; tetrafluoroborate; hexafluorophosphate; hexachloroantimonate or tetraphenylborate.

Preferably in a compound of formula (III), X is a non-coordinating anion, typically a sulfonate, in particular trifluoromethanesulfonate. Preferably an Au(I) based catalyst, having formula (III), wherein X is a non-coordinating or slightly coordinating anion, as defined above, can be produced in situ by reacting an Au(I) compound of formula (IV)

P(Ra)₃AuX₁ (IV)

wherein Ra is as defined above, and X₁ is a coordinating anion, for example chloride, with a silver salt of formula (V)

AgY (V)

wherein Y is slightly coordinating or non-coordinating, organic or inorganic anion as defined above, in particular trifluoromethanesulfonate.

X₁ as coordinating anion has the same values of X as coordinating anion defined above, preferably chloride.

Preferably compound of formula (IV) is Ph₃PAuCl and a compound of formula (V) is AgCF₃SO₃, so that the catalyst of formula (III) obtainable in situ is Ph₃PAuCF₃SO₃, wherein Ph is phenyl.

The molar ratio of a compound of formula (IV), for example PPh₃AuCl, compared to the alkyne of formula (II) can be comprised between about 0.01% and about 2%; preferably between about 0.1% and about 1%.

A strong protic acid can be a strong organic or inorganic acid.

For example a strong inorganic acid can be sulphuric acid; a hydrohalic acid, preferably hydrochloric acid; nitric or perchloric acid; preferably sulphuric acid. Typically such acid is used in the form of an aqueous solution thereof.

A strong organic acid can be for example a C₁-C₆ alkylsulfonic acid, a C₃-C₁₀ cycloalkylsulfonic acid; a C₆-C₁₀ bicycloalkylsulfonic or aryl-sulfonic acid, optionally substituted by one or more substituents, preferably from 1 to 9, independently chosen from halogen, in particular fluorine; preferably methanesulfonic, trifluoromethanesulfonic, camphorsulfonic or p-toluenesulfonic acid. More preferably, the strong protic acid is methanesulfonic or p-toluenesulfonic acid, in particular in hydrate form.

The molar ratio of the strong protic acid compared to the alkyne of formula (II) can be comprised between about 1 and about 7, preferably between about 2 and about 5.

The molar ratio of water in the reaction mixture compared to the alkyne of formula (II) is at least stoichiometric.

If desired, the hydration reaction of an alkyne of formula (II) to obtain a ketone of formula (I) can be carried out in a polar aprotic or protic solvent. Typically an aprotic polar solvent can be dimethylformamide, dimethylacetamide, acetonitrile, dimethylsulfoxide; an ether, for example diethylether, methyl-tert-butyl ether, tetrahydrofuran or dioxane; a chlorinated solvent, for example, dichloromethane, dichloroethane, chloroform or chlorobenzene; a C₁-C₆ alkyl ester, for example ethyl or methyl acetate; or a C₃-C₁₂ ketone, for example acetone, methylethylketone, methylisobutylketone. More preferably said solvent is tetrahydrofuran.

A protic polar solvent typically can be water or a C₁-C₁₈ alkanol, preferably a C₁-C₅ alkanol, more preferably methanol, ethanol, or isopropanol, or a mixture thereof with water; a weak C₁-C₅ carboxylic acid, preferably acetic acid; or a mixture of two or more, preferably two or three of said solvents. More preferably said solvent is ethanol.

The reaction is preferably carried out by first dissolving the alkyne compound of formula (II) in a polar protic or aprotic solvent, as defined above. The molar concentration of the alkyne of formula (II) in said solution can be approximately comprised between 0.1 and 10 M, preferably between about 0.3 and about 3M.

The reaction can be carried out at a temperature comprised between about 0° C. and the reflux temperature of the reaction mixture, preferably between about 20 and about 50° C.

The conversion of a compound of formula (I) into another compound of formula (I), for example the conversion of the carboxylic ester into the free acid, or vice versa, can be carried out according to known methods.

A so obtained compound of formula (I), wherein R is hydrogen, can be converted into fexofenadine of formula (VI) or a salt thereof

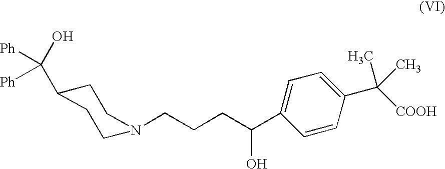

according to known methods, for example as disclosed in EP 1616861.

Therefore the invention also provides a process for the preparation of fexofenadine, or a salt thereof, further comprising the reduction of a so obtained keto compound of formula (I), wherein R is H,

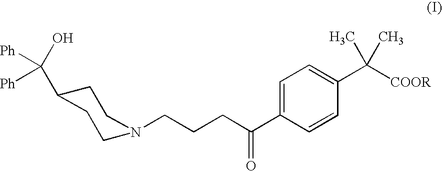

to obtain fexofenadine and, if desired, its conversion into a salt thereof. A fexofenadine salt is for example a pharmaceutically acceptable salt thereof, in particular the hydrochloride.

The following examples illustrate the invention.

### EXAMPLE I

### Synthesis of methyl 4-{[4-(4-hydroxyphenylmethyl)-1-piperidinyl]-1-oxobutyl}-α,α-dimethylbenzenacetate (I, R=Me)

The alkyne of formula II (R=Me) (200 mg, 0.40 mmol) is dissolved in absolute ethanol (0.8 ml) and then treated with monohydrate p-toluenesulfonic acid (383 mg, 2.02 mmol). After 10 minutes, Ph₃AuCl (4 mg, 0.0081 mmol, 2% molar) and AgCF₃SO₃ (2 mg, 0.0081 mmol, 2% molar) are added. After 48 h, at room temperature, the ethanol is evaporated under reduced pressure and the residue is taken up with ethyl acetate and basified till pH 10-11 with a NaHCO₃ solution. The phases are separated, and the aqueous one is extracted with ethyl acetate. The organic phases are collected, dried with Na₂SO₄, filtered off and evaporated under reduced pressure. The crude reaction is purified by flash chromatography (acetate/methanol 9:1 as eluent) and 198 mg of title keto compound are obtained as a white solid, with a yield of 95%.

¹H NMR (400 MHz, CDCl₃) δ ppm: 7.94 (d, J=8.5 Hz, 2H), 7.49 (d, J=8.0 Hz, 4H), 7.43 (d, J=8.6 Hz, 2H), 7.34-7.29 (m, 4H), 7.22-7.17 (m, 2H), 3.65 (s, 3H), 3.04-2.95 (m, 4H), 2.49-2.42 (m, 3H), 2.05-1.92 (m, 4H, 1H exchanges with D₂O), 1.62 (s, 6H), 1.52-1.41 (m, 4H).

### EXAMPLE 2

### Synthesis of 4-{[4-(4-hydroxyphenylmethyl)-1-piperidinyl]-1-oxobutyl}-α,α-dimethylbenzenacetic acid (I, R=H)

The alkyne of formula II (R=H) (2.0 g, 4.16 mmol) is dissolved in ethanol (8 ml) and then treated with monohydrate p-toluenesolfonic acid (3.9 g, 20.8 mmol). After 10 minutes Ph₃PAuCl (41 mg, 0.083 mmol, 2% molar) and AgCF₃SO₃ (21 mg, 0.083 mmol, 2% molar) are added. After 24 h, at room temperature, the ethanol is evaporated off under reduced pressure and the residue is taken up with THF. The reaction mixture is heated and basified with a NaOH solution. The phases are separated and the organic one is treated with acetic acid. The white crystallized solid is filtered off and dried. 1.97 g of the title product (I) are obtained with a yield of 95%.

¹NMR (300 MHz, CDCl₃) δ ppm: 7.70 (d, J=8.4 Hz, 2H), 7.52-7.46 (m, 6H), 7.32-7.26 (m, 4H), 7.20-7.15 (m, 2H), 3.47 (s, 1H), 3.37 (m, 2H), 2.74-2.63 (m, 4H), 2.55 (m, 1H), 2.39-2.32 (m, 2H), 2.03-1.78 (m, 4H), 1.58-1.52 (m, 8H).

### EXAMPLE 3

### Synthesis of 4-{[4-(4-hydroxyphenylmethyl)-1-piperidinyl]-1-oxobutyl}-α,α-dimethylbenzenacetic acid (I, R=H)

The alkyne of formula II (R=H) (50.0 g, 0.104 mol) is dissolved in tetrahydrofuran (225 ml) and treated with 62.5% sulfuric acid (81.5 g, 0.519 mol), dropwise added. After 30 minutes water (10 g), Ph₃PAuCl (1.03 g, 2.1 mmol) and AgNO₃ (0.36 g, 2.1 mmol) are added. The reacting mixture is heated to 45° C. and maintained at the same temperature for 18 h under stirring. The mixture is then diluted with water and basified with a 30% NaOH solution at around 40-50° C. The mixture is maintained under reflux for 16 h under stirring and then filtered on charcoal. The solution is neutralized by addition of acetic acid and cooled to 5-10° C. under stirring. The crystallized solid is filtered off and dried. Title product (I) is obtained (41.0 g) with a 79% yield and more than 99% purity calculated by HPLC.

¹H NMR (300 MHz, CDCl) δ ppm: 7.70 (d, J=8.4 Hz, 2H), 7.52-7.46 (m, 6H), 7.32-7.26 (m, 4H), 7.20-7.15 (m, 2H), 3.47 (s, 1H), 3.37 (m, 2H), 2.74-2.63 (m, 4H), 2.55 (m, 1H), 2.39-2.32 (m, 2H), 2.03-1.78 (m, 4H), 1.58-1.52 (m, 8H).

1. A process for the preparation of a compound of formula (I) 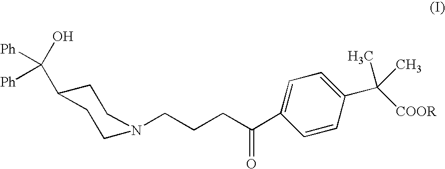
wherein Ph is a phenyl ring and R is hydrogen or C₁-C₆ alkyl group, comprising reacting an alkyne of formula (II) 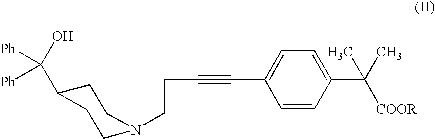
wherein Ph and R are as defined above, with a strong protic acid in the presence of water and an Au(I) based catalyst and, if desired, converting a compound of formula (I) into another compound of formula (I).

2. A process according to claim 1, wherein the strong protic acid is chosen from sulphuric, hydrochloric, perchloric, methanesulfonic, camphorsulfonic, trifluoromethanesulfonic and p-toluenesulfonic acid.

3. A process according to claim 1, wherein the molar amount of the strong protic acid to the alkyne of formula (II) is comprised between about 1 and about 5.

4. A process according to claim 1, wherein the molar amount of water in the reaction mixture to the alkyne of formula (II) is at least stoichiometric.

5. A process according to claim 1, wherein an Au(I) based catalyst is a compound having the following formula (III)
P(Ra)₃AuX (III)
wherein each of Ra, being the same or different, is a straight or branched C₁-C₃₀ alkyl group; a C₃-C₁₀ cycloalkyl ring; an aryl or heteroaryl ring; and X is a coordinating or non-coordinating, organic or inorganic anion.

6. A process according to claim 5, wherein the anion X is a halide, an azide, a sulphate, a nitrate, a sulfonate preferably trifluoromethanesulfonate, a sulphinate, a C₁-C₆ alcoholate, a phenate, a carboxylate, a perchlorate, a tetrafluoroborate, a hexafluorophosphate, a hexacloroantimoniate or a tetraphenylborate residue.

7. A process according to claim 5, wherein the compound of formula (III) is Ph₃PAuCF₃SO₃, wherein Ph is phenyl.

8. A process according to claim 5, wherein the compound of formula (III) is prepared in situ by reaction of an Au(I) compound of formula (IV)
P(Ra)₃Au X₁ (IV)
wherein Ra is as defined in claim 5, and X₁ is a coordinating anion, with a silver salt of formula (V)
AgY (V)
wherein Y is a slightly coordinating or non-coordinating, organic or inorganic anion.

9. A process according to claim 8, wherein the coordinating anion X₁ is chloride and the anion Y is trifluoromethanesulfonate.

10. A process according to claim 8, wherein the molar amount of a compound of formula (IV), to the alkyne of formula (II) is comprised between about 0.01% and about 2%.

11. A process according to claim 1, further comprising the reduction of a keto compound of formula (I), wherein R is H, 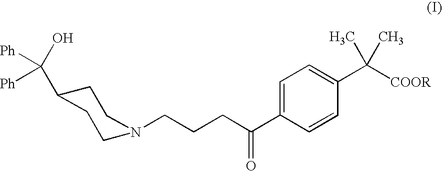
to obtain fexofenadine and, if desired, its conversion into a salt thereof.
